(19) United States
(12) Patent Application Publication
(10) Pub. No.: US 2016/0174937 A1
(43) Pub. Date: Jun. 23, 2016

(54) WIRELESS ULTRASOUND PROBE

(71) Applicant: General Electric Company, Schenectady, NY (US)

(72) Inventors: Tarun Bakshi, Bangalore (IN); Menachem Halmann, Wauwatosa, WI (US)

(21) Appl. No.: 14/712,282

(22) Filed: May 14, 2015

(30) Foreign Application Priority Data
Dec. 23, 2014 (IN) 6493/CHE/2014

(51) Int. Cl. A61B 8/00; A61B 19/00; A61B 8/08

(52) U.S. Cl. CPC A61B 8/4472; A61B 8/565; A61B 8/467; A61B 8/4477; A61B 8/52; A61B 19/44

(57) ABSTRACT

A wireless ultrasound probe is disclosed. The wireless ultrasound probe includes an image capturing unit for capturing image data, a processor configured to end a scan session once image data associated with an anatomy of a patient is captured and start a new scan session for capturing image data associated to an anatomy of another patient, and a wireless communication unit configured to wirelessly communicate with at least one of storage and an ultrasound imaging system. The wireless communication unit facilitates transfer of the image data to at least one of storage and the ultrasound imaging system.

### FIELD OF THE INVENTION

The subject matter disclosed herein relates to performing ultrasound imaging on patient. More specifically the subject matter relates to an ultrasound probe having wireless capability for capturing image data associated with patient's anatomy.

### BACKGROUND OF THE INVENTION

Medical imaging systems such as ultrasound imaging systems are used in different applications to image different regions or areas (e.g. different organs) of patients or other objects. For example, an ultrasound imaging system may be utilized to generate an image of organs, vasculature, heart, or other portions of the body. Ultrasound imaging systems are generally located at a medical facility, for example, a hospital or an imaging center. However, not all people have access to a medical facility. In particular, individuals at nursing homes, under home care, or in rural areas may not be capable of attending a medical facility for ultrasound imaging

An ultrasound probe is used for capturing image data associated with an anatomy of a patient. The image data can be processed to generate images of the anatomy. The ultrasound probe is used for scanning a patient one by one which becomes practically difficult. Ultrasound imaging system is a single unit with one or more ultrasound probes that needs to be moved from one location to another based on requirement. This becomes cumbersome as the whole unit needs to be moved to different locations for instance from one floor to another floor of a building. Moreover at an instance the ultrasound probe can be used to perform scan on one patient at a time. Accordingly, a need exists to an improved ultrasound probe for capturing image data of the patient in a convenient manner.

### SUMMARY OF THE INVENTION

The object of the invention is to provide an ultrasound probe for capturing image data associated with a patient to another, which overcomes one or more drawbacks of the prior art. This is achieved by a wireless ultrasound probe for capturing image data associated with an anatomy of a patient as defined in the independent claim.

One advantage with the disclosed invention is that a wireless ultrasound probe can be used to capture image data associated with the patient's anatomy conveniently as this can be moved away from an ultrasound imaging system. The wireless ultrasound probe can be used to capture the image data from the patients one by one. When the wireless ultrasound probe captures the image data from a patient it is automatically stored in one of storage and an ultrasound imaging system.

In an embodiment a wireless ultrasound probe is disclosed. The wireless ultrasound probe includes an image capturing unit for capturing image data associated with an anatomy of a patient; a processor configured to end a scan session once image data associated with an anatomy of a patient is captured and start a new scan session for capturing image data associated to an anatomy of another patient; and a wireless communication unit configured to wirelessly communicate with one or more of storage and an ultrasound imaging system. The wireless communication unit facilitates transfer of the image data to one or more of the storage and the ultrasound imaging system.

In another embodiment an ultrasound imaging system is disclosed. The ultrasound imaging system includes an image processing unit for processing a plurality of images associated to anatomy of patients; a plurality of wireless ultrasound probes configured to wirelessly communicate with the image processing unit. The wireless ultrasound probe of the plurality of wireless ultrasound probes includes an image capturing unit for capturing image data associated to an anatomy of a patient; a processor configured to end a scan session once image data associated with an anatomy of a patient is captured and start a new scan session for capturing image data associated to an anatomy of another patient; and a wireless communication unit configured to wirelessly communicate with an image processing unit, the wireless communication unit facilitate transfer of the image data to the image processing unit.

A more complete understanding of the present invention, as well as further features and advantages thereof, will be obtained by reference to the following detailed description and drawings.

### BRIEF DESCRIPTION OF THE DRAWINGS

FIG. 1 is a schematic illustration of an ultrasound imaging system in accordance with an embodiment in accordance with an embodiment;

FIG. 2 is schematic illustration of a wireless ultrasound probe according to an embodiment;

FIG. 3 illustrates an exemplary environment wherein the wireless ultrasound probe of FIG. 2 operates;

FIG. 4 is a schematic illustration of wireless ultrasound probe communicating with different ultrasound imaging system in different wireless coverage areas;

FIG. 5 illustrates a wireless ultrasound probe communicating with a portable ultrasound imaging device according to an embodiment; and

FIG. 6 illustrating multiple wireless ultrasound probes wirelessly communicating with an ultrasound imaging system according to an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical and other changes may be made without departing from the scope of the embodiments. The following detailed description is, therefore, not to be taken as limiting the scope of the invention.

As discussed in detail below, embodiments of the invention including a wireless ultrasound probe is disclosed. The wireless ultrasound probe includes an image capturing unit for capturing image data associated with an anatomy of a patient; a processor configured to end a scan session once image data associated with an anatomy of a patient is captured and start a new scan session for capturing image data associated to an anatomy of another patient; and a wireless communication unit configured to wirelessly communicate with one or more of storage and an ultrasound imaging system. The wireless communication unit facilitates transfer of the image data to one or more of the storage and the ultrasound imaging system.

FIG. 1 is a schematic illustration of an ultrasound imaging system 100 in accordance with an embodiment. The ultrasound imaging system 100 includes a transmitter 102 that transmits a signal to a transmit beam-former 104 which in turn drives transducer elements 106 within a transducer array 108 to emit pulsed ultrasonic signals into a structure, such as a patient (not shown). A probe 110 includes the transducer array 108, the transducer elements 106 and probe/SAP electronics 112. The probe/SAP electronics 112 may be used to control the switching of the transducer elements 106. The probe/SAP electronics 112 may also be used to group the transducer elements 104 into one or more sub-apertures. A variety of geometries of transducer arrays may be used. The pulsed ultrasonic signals are back-scattered from structures in the body, like blood cells or muscular tissue, to produce echoes that return to the transducer elements 106. The echoes are converted into electrical signals, or ultrasound data, by the transducer elements 106 and the electrical signals are received by a receiver 114. For purposes of this disclosure, the term ultrasound data may include data that was acquired and/or processed by an ultrasound system. The electrical signals representing the received echoes are passed through a receive beam-former 116 that outputs ultrasound data. A user interface 118 may be used to control operation of the ultrasound imaging system 100, including, to control the input of patient data, to change a scanning or display parameter, and the like.

The ultrasound imaging system 100 also includes a processor 120 to process the ultrasound data and generate frames or images for display on a display screen 122. The processor 120 may be adapted to perform one or more processing operations according to a plurality of selectable ultrasound modalities on the ultrasound data. Other embodiments may use multiple processors to perform various processing tasks. The processor 120 may also be adapted to control the acquisition of ultrasound data with the probe 110. The ultrasound data may be processed in real-time during a scanning session as the echo signals are received. An embodiment may update the displayed ultrasound image at a rate of more than 20 times per second. The images may be displayed as part of a live medical image. For purposes of this disclosure, the term “live medical image” is defined to include a dynamic image that updates as additional frames of ultrasound data are acquired. For example, ultrasound data may be acquired even as images are being generated based on previously acquired data and while a live medical image is being displayed. Then, according to an embodiment, as additional ultrasound data are acquired, additional frames or images generated from more-recently acquired ultrasound data are sequentially displayed. Additionally or alternatively, the ultrasound data may be stored temporarily in a buffer (not shown) during a scanning session and processed in less than real-time in a live or off-line operation. Some embodiments of the invention may include multiple processors (not shown) to handle the processing tasks. For example, a first processor may be utilized to demodulate and decimate the ultrasound signal while a second processor may be used to further process the data prior to displaying an image. It should be appreciated that other embodiments may use a different arrangement of processors.

The images may be obtained or acquired at different scanning planes based on an imaging procedure that is performed. The imaging procedure may be for example fetal biometry and wellbeing assessment, fetal anatomical survey, abdominal scanning, and cardiac scanning. Considering the case of fetal anatomical survey, various imaging parameters may be accessed such as head, face, neck, chest/heart, abdomen, skeletal, placenta and umbilical cord. The imaging parameters in this case are body parts and/or organs of a fetus. The images are acquired from different scanning planes such as but not limited to, an axial plane, a transventricular plane, a transthalamic plane, a transcerebellar plane, a coronal plane, a sagittal plane and a mid-sagittal plane. The imaging parameters vary depending on the imaging procedure selected by the user. For instance to perform fetal biometry and wellbeing assessment, imaging parameters such as a biparietal diameter, a head circumference, an abdominal circumference and a femur diaphysis length may be analyzed using the ultrasound imaging system 100.

Still referring to FIG. 1, the ultrasound imaging system 100 may continuously acquire ultrasound data at a frame rate of, for example, 20 Hz to 150 Hz. However, other embodiments may acquire ultrasound data at a different rate. A memory 124 is included for storing processed frames of acquired ultrasound data that are not scheduled to be displayed immediately. In an exemplary embodiment, the memory 124 is of sufficient capacity to store at least several seconds worth of frames of ultrasound data. The frames of ultrasound data are stored in a manner to facilitate retrieval thereof according to its order or time of acquisition. As described hereinabove, the ultrasound data may be retrieved during the generation and display of a live medical image. The memory 124 may comprise any known data storage medium.

In various embodiments of the present invention, ultrasound information may be processed by other or different mode-related modules (e.g., B-mode, Color Doppler, power Doppler, M-mode, spectral Doppler anatomical M-mode, strain, strain rate, and the like) to form 2D or 3D data sets of image frames and the like. For example, one or more modules may generate B-mode, color Doppler, power Doppler, M-mode, anatomical M-mode, strain, strain rate, spectral Doppler image frames and combinations thereof, and the like. The image frames are stored and timing information indicating a time at which the image frame was acquired in memory may be recorded with each image frame. The modules may include, for example, a scan conversion module to perform scan conversion operations to convert the image frames from Polar to Cartesian coordinates. A video processor module may be provided that reads the image frames from a memory and displays the image frames in real time while a procedure is being carried out on a patient. A video processor module may store the image frames in an image memory, from which the images are read and displayed. The ultrasound imaging system 100 shown may comprise a console system, or a portable system, such as a hand-held or laptop-type system.

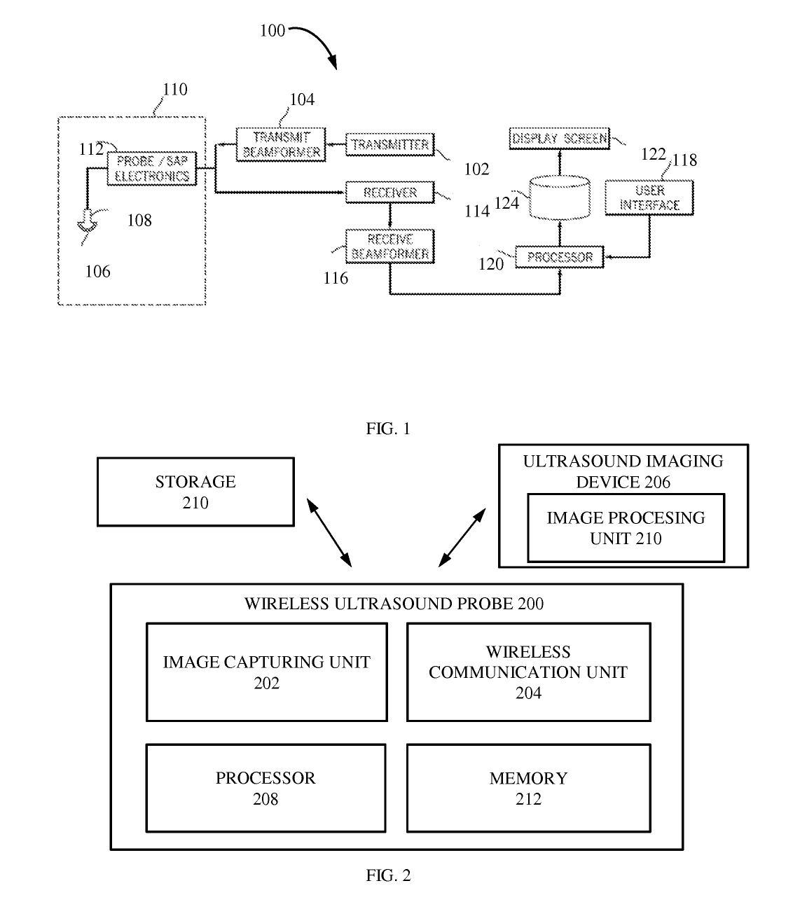

FIG. 2 illustrates a wireless ultrasound probe 200 according to an embodiment. The wireless ultrasound probe 200 includes an image capturing unit 202 for capturing image data (i.e. ultrasound data) associated with an anatomy of a patient. The image data can be processed to generate one or more images. The image data may be obtained or collected by a transducer array. The transducer array includes multiple transducer elements that are configured to transmit ultrasonic signals that can be processed to generate the image data. A wireless communication unit 204 is configured to wirelessly communicate with an ultrasound imaging device 206. The wireless communication unit 204 facilitates transfer of the image data to the ultrasound imaging device 206. The wireless communication unit 204 may be configured over a wi-fi network. In another embodiment the wireless communication unit 204 may operate based on Bluetooth® technique. The wireless ultrasound probe 200 may be configured or registered with the ultrasound imaging device 206 over a wireless network for example the wi-fi network. The wireless communication unit 204 may send configuration information for connecting the wireless ultrasound probe 200 to the ultrasound imaging device 206. In an embodiment the wireless communication unit 204 may send connection request to the ultrasound imaging device 206 and it may be authenticated based on configuration information for connecting the wireless ultrasound probe 200.

The image data may be captured from the patient in a scan session. The scan session may refer to a scanning procedure performed on the patient. In an embodiment the scanning procedure may be a particular scan performed on an anatomy of the patient. The anatomy may be for instance an abdomen, a heart and so on. The scanning procedure may be different for different anatomies. The scanning procedure may be in another instance an obstetric imaging, an abdominal imaging and a cardiac imaging. The scanning procedure may be a sub-step in a scanning process. For instance a sub-step in an abdominal imaging procedure.

Once the scan session is over, the wireless ultrasound probe 200 can be used to perform scan on another patient. The scan performed on this patient is a new scan session. The new scan session may involve multiple scans to be performed on the patient to analyze the patient's anatomy. Each of these scan may have different imaging presets. For example in abdominal imaging there may be different presets based on organ type (e.g. liver, kidney etc.), and a subject type (e.g. normal, difficult). Further in obstetric imaging a preset may be based on trimester type for example a first semester and a second semester. The imaging presets described herein is few exemplary ones and it may be envisioned that multiple other imaging presets may be present in the ultrasound imaging device 206.

The imaging preset for performing a particular scan may be set in the wireless ultrasound probe 200 by a processor 208. The processor 208 changes the imaging preset based on user input. The user may be a person operating the wireless ultrasound probe 200 i.e. an ultrasound expert or technician, a doctor and so on. The user input may be obtained in response to activation of a button in the wireless ultrasound probe 200. In another instance user input may be a gesture. The gesture may be a hand gesture. Other gestures may include but are not limited to, shaking the wireless ultrasound probe 200, any movement of wireless ultrasound probe 200 in a particular manner, and rotational movement of the wireless ultrasound probe 200. The gesture when identified, the processor 208 changes an imaging preset. Even though only imaging preset is described herein to be modified or changed it may be envisioned that other imaging configurations for performing a scanning can be also changed based on gestures or a user input. The imaging configuration may include various parameters such as frequency, a speckle reduction imaging, time gain compensation, scan depth, scan format, image frame rate, field of view, focal point, scan lines per image frame, number of ultrasound beams and pitch of the transducer elements.

In an embodiment the processor 208 is further configured to a set a rounds mode. In the rounds mode a scan session is closed and a new scan session starts in response to a trigger event. The closed scan session and the new scan session are associated with one patient. For example in a rounds mode the ultrasound probe 200 may be used to perform a scanning procedure on a patient. The scanning procedure may be abdominal scanning. Then based on the trigger event another scanning procedure may be performed on the same patient. The trigger event may be based on the user input. The trigger event may be in response to the processor 208 receiving and processing an identification code associated with the patient. The identification code may be read from but not limited to an RFID tag or a bar code tag associated with the patient. The RFID tag or the bar code tag may include identification information that may be a unique ID of the patient. In an embodiment the ultrasound probe 200 may have a reader (i.e. an inbuilt reader) for reading the identification information of the patient from the RFID tag or the bar code tag. Alternatively the ultrasound probe 200 may have another reader which can be used for reading the identification information. In another embodiment the trigger event may occur based on a user input. The user input may be by activation of a button/switch provided in the ultrasound probe 200. Further the user input in another scenario may be by providing gesture by the user that can be detected and processed by the ultrasound probe 200. The gesture may be for instance but not limited to shaking the ultrasound probe 200 in a particular manner.

As the ultrasound probe 200 is used to scan multiple patients, a previous patient on whom a scanning procedure was already done may need to have another round of scanning procedure. In such situations when the ultrasound probe 200 is brought to the patient, the processor 208 may be configured to determine the presence of a previous scan session of the patient. Here initially the patient identification code may be identified and accordingly history of the scanning procedure performed on the patient may be retrieved. The information including the scanning procedures performed on each patient may be stored in the storage 210 or the ultrasound imaging device 206. In an embodiment when a scanning procedure is completed, information associated with the scanning procedure is linked to the identification code of the patient is stored. The processor 208 may link the information of the scanning procedure and the identification code and then send this to the storage 210 or the ultrasound imaging device 206. Alternatively the process of linking the information of the scanning procedure and the identification code is performed in the ultrasound imaging device 206. Further imaging presets used during the scanning procedure may be also linked to the identification code and stored. It may be various embodiments may employ different methods of linking the information of the scanning procedure and the identification code of the patient and ways of storing these information which are within the scope of this disclosure.

So when the ultrasound probe 200 is used to perform another round of scanning on a patient on whom a scanning procedure was previously performed, The processor 208 determines the presence of a previous scan session on the patient and may set one or more imaging presets of the previous scan session in response to starting a new scan session on the patient. These imaging presets may be used to perform a scanning procedure in the new scan session if required. Otherwise the user can set the ultrasound probe 200 to use different imaging presets to perform the scanning procedure. Further the processor 208 may be configured to present scan data and image data associated with the previous scan session. The scan data may include any results and interpretation of the user of the previous scan session. The image data may be medical images (i.e. ultrasound images) captured from the patient. Thus the ultrasound probe 200 can be used for performing different rounds of scanning procedure on multiple patients. This also facilitates recollecting the scan data and the image data from a previous scan session on a patient and considers a new scan session as a follow up round to the previous scan session.

When each scan session is completed the wireless communication unit 204 transfers the image data to the ultrasound imaging device 206. The image data may be processed to generate images and stored in the ultrasound imaging device 206. Thus the wireless ultrasound probe 200 can be used to perform different scan sessions on multiple patients and push or transfer the image data to the ultrasound imaging device 206. In another embodiment the wireless ultrasound probe 200 may be configured to process the image data to generate images i.e. ultrasound images or medical images. In this embodiment the processor 208 receives the image data and processes it to generate the ultrasound images. The ultrasound images are then wirelessly transferred to the ultrasound imaging device 206 using the wireless communication unit 204. Moreover in another scenario, the image data from the wireless ultrasound probe 200 may be transferred to storage 210. In another embodiment the ultrasound images may be transferred to the storage 210. The storage 210 may be a database that can store the image data or the ultrasound images. The storage can have capability to wirelessly communicate with the wireless ultrasound probe 200. In an embodiment the storage may be part of an ultrasound imaging system. The image data and the ultrasound images if generated by the wireless ultrasound probe 200 may be stored in a memory 212. Here the image data and the ultrasound images may be transferred to the ultrasound imaging device 206 over the wireless network.

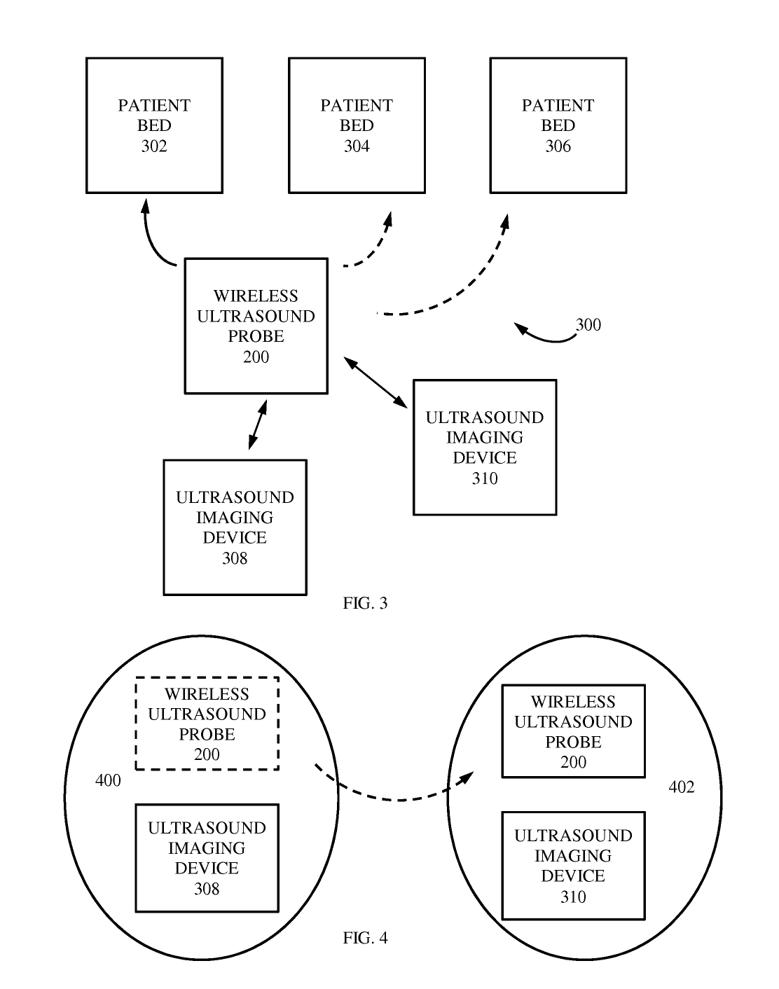

FIG. 3 illustrates an exemplary environment 300 wherein the wireless ultrasound probe 200 operates. The environment 300 includes multiple patient beds such as a patient bed 302, a patient bed 304 and a patient bed 306. Three patient beds illustrated herein is exemplary and thus the environment 300 may include more than three patient beds. The patient beds may have infants or newborns or pregnant women that need to be scanned. The ultrasound probe 200 may be used to perform ultrasound scan on a patient in the patient bed 302. The ultrasound probe 200 may have an imaging preset already configured and the user may have already decided a type of scanning procedure for example abdominal imaging, obstetric imaging, cardiac imaging and so on to be performed. The image data captured from the patient may be transferred to storage or an ultrasound imaging system wirelessly. This may be a scan session for scanning the patient in the patient bed 302. Thereafter the wireless ultrasound probe 200 may be used to scan a patient on the patient bed 304. The image data captured from the patient is wirelessly transferred to the ultrasound imaging system for further processing so as to move to the next patient bed 306. So the user of the wireless ultrasound probe 200 can shift between the patients in the patient beds 302, 304 and 306 without viewing and checking the ultrasound images captured. Thus the user can attend and perform imaging on the patients faster and no delay is experienced in the scanning procedure. Once all the patients are attended in the patient beds the ultrasound images generated by the ultrasound imaging system can be viewed by the ultrasound technician or a doctor or the user. In another scenario the ultrasound images generated from the image data may be viewed by a technician or doctor at real time and provide feedback to the user of the wireless ultrasound probe 200. This feedback enables the user to correct the scanning performed at this stage avoiding repetition of another cycle of scanning procedure on a patient.

In an alternative embodiment display units may be allocated to each patient bed for presenting the ultrasound images. For instance the image data captured from a patient on the patient bed 302 may be processed at the ultrasound imaging system and ultrasound images transferred to a display unit associated with the patient bed 302 in real time. The bandwidth of a wireless communication network for live streaming of the ultrasound images to the display unit from the ultrasound imaging system is high. Wireless network booster may be placed in different locations so that the wireless ultrasound probe 200 can connected with the ultrasound imaging system from a long distance.

The wireless ultrasound probe 200 may communicate wirelessly with multiple ultrasound imaging systems such as an ultrasound imaging system 308 and an ultrasound imaging device 310. These ultrasound imaging systems may be functionally similar and operate in the same manner for processing the image data received from the wireless ultrasound probe 200. In another embodiment the ultrasound images generated in the wireless ultrasound probe 200 may be transferred directly to the ultrasound imaging system 308 and the ultrasound imaging device 310. The wireless channels for instance Wi-Fi channels used by the wireless ultrasound probe 200 for communicating with the ultrasound imaging system 308 and the ultrasound imaging device 310 may be different. In an embodiment the wireless ultrasound probe 200 may be only able to communicate with on ultrasound imaging system at a time. In another embodiment the wireless ultrasound probe 200 may be capable of communicating with both the ultrasound imaging systems at the same time and hence can transfer ultrasound images or image data simultaneously. The ultrasound imaging system 308 and the ultrasound imaging device 310 may be located at different locations which within the wireless coverage of the wireless ultrasound probe 200.

Considering a hospital environment where there will be numerous beds and patients, the wireless ultrasound probe can be moved around in the hospital for performing scanning procedure. Ultrasound imaging systems may be placed in one or more locations for processing the image data received from the wireless ultrasound probe. The doctor or technician may be viewing the ultrasound images processed in the ultrasound imaging system which has a display unit. In another scenario the ultrasound images may be generated from the image data received and stored for review at a later time. The ultrasound probe can be carried by the user to different locations in the hospital.

The ultrasound imaging system 308 and the ultrasound imaging device 310 may be in different wireless coverage area as shown in FIG. 4. The ultrasound imaging system 308 may be in a wireless coverage area 400 and the ultrasound imaging system 402 may be in another wireless coverage area 402. Thus the wireless network and the wireless channel for communication in the wireless coverage areas 400 and 402 are different. So the wireless ultrasound probe 200 may be initially performing scanning procedures within the wireless coverage area 400 and thus may be connected to the ultrasound imaging system 308. The image data captured by the wireless ultrasound probe 200 at this instance are wirelessly transferred and processed at the ultrasound imaging system 308. The wireless ultrasound probe 200 may move to the wireless coverage area 402 when it moves around in the hospital. The wireless ultrasound probe 200 may need to get connected with the ultrasound imaging device 310. Then the wireless communication between the wireless ultrasound probe 200 and the ultrasound imaging device 310 may be established so that image data captured now can be transferred to the ultrasound imaging device 310 for processing and storage.

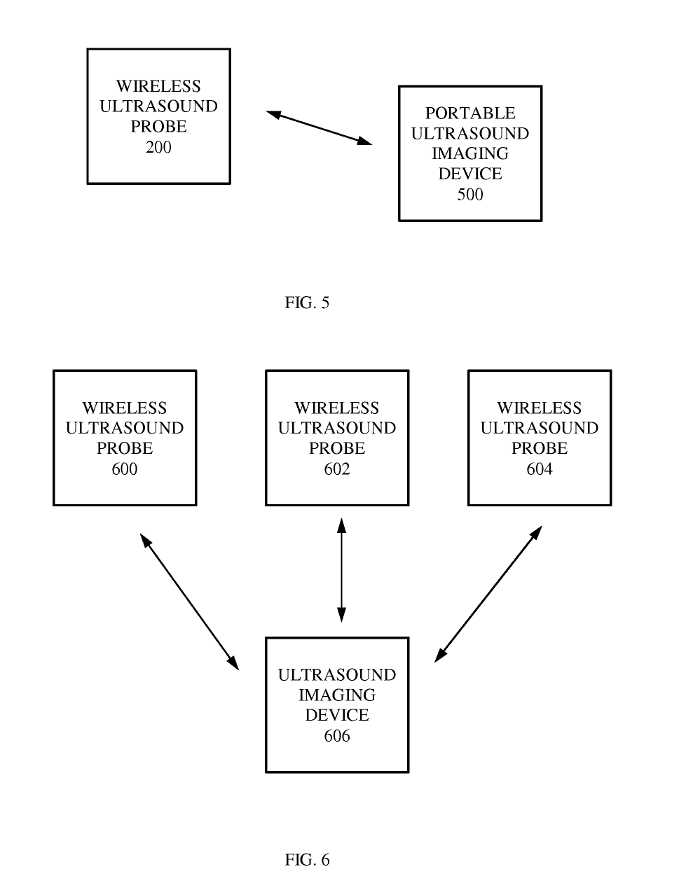

FIG. 5 illustrates the wireless ultrasound probe 200 communicating with a portable ultrasound imaging device 500 according to an embodiment. The portable ultrasound imaging device 500 may be a portable ultrasound device that has wireless capability and communicate over a wireless network with the wireless ultrasound probe 200. The portable ultrasound imaging device 500 may be a tablet device in an embodiment. The wireless ultrasound probe 200 may wirelessly transfer image data or ultrasound images to the portable ultrasound imaging device 500. The portable ultrasound imaging device 500 may then process the image data to generate the ultrasound images. In another embodiment the portable ultrasound imaging device 500 may be an application that may be function in a portable device or a tablet device or a mobile device and so on. The image data may be transferred over a Wi-Fi network between the portable device and the wireless ultrasound probe 200. So for example in a hospital, if the doctor needs to see the ultrasound images of a patient then he may be able to view them in the mobile device. In another instance the mobile device may be configured to process the image data received from the wireless ultrasound probe 200 to generate the ultrasound images.

Turning now to FIG. 6 illustrating a wireless ultrasound probe 600, a wireless ultrasound probe 602, and a wireless ultrasound probe 604 wirelessly communicating with an ultrasound imaging device 606 according to an embodiment. The wireless ultrasound probes 600, 602 and 604 can establish communication with the ultrasound imaging device 606. The communication may be over a wireless network such as Wi-Fi network and a Bluetooth® network. The wireless ultrasound probe 600 may capture image data from the patient and wirelessly transfer the image data to the ultrasound imaging device 606. The ultrasound imaging device 606 can process the image data to generate ultrasound images. In an embodiment the wireless ultrasound probe 600, the wireless ultrasound probe 602, and the wireless ultrasound probe 604 can communicate simultaneously with the ultrasound imaging device 606 to transfer image data captured by respective probes. The wireless ultrasound probes may communicate over different wireless channels to the ultrasound imaging device 606. The ultrasound imaging device 606 may communicate only with one wireless ultrasound probe at an instance. In a hospital environment these wireless ultrasound probes may be located at different locations.

From the foregoing, it will appreciate that the above disclosed a patient table assembly for transferring a patient in a hospital environment provide numerous benefits to healthcare enterprises, such as improved way of transferring the patient from one location to another which less labor some for the user. Further as the flexible table top of the patient table assembly disclosed here is made of cloth material the flexible table top can be washed and cleaned easily in case there are any spillovers and becomes dirty when any procedures are done of the patient.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

This written description uses examples to disclose the invention, including the best mode, and also to enable any person skilled in the art to practice the invention, including making and using any computing system or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A wireless ultrasound probe comprising:
an image capturing unit for capturing image data;
a processor configured to end a scan session once image data associated with an anatomy of a patient is captured and start a new scan session for capturing image data associated to an anatomy of another patient; and
a wireless communication unit configured to wirelessly communicate with at least one a storage and an ultrasound imaging system, the wireless communication unit facilitate transfer of the image data to at least one of the storage and the ultrasound imaging device.

2. The wireless ultrasound probe of claim 1, wherein the wireless communication unit is configured to communicate over one of a Wi-Fi network and a Bluetooth network.

3. The wireless ultrasound probe of claim 1, wherein the scan session ends based on user input, the user input is received based on at least one of activation of a switch in the wireless ultrasound probe, a gesture and a motion gesture of the wireless ultrasound probe.

4. The wireless ultrasound probe of claim 3, wherein the image data captured in the scan session is transferred to at least one the storage and the ultrasound imaging device prior to starting the new scan session.

5. The wireless ultrasound probe of claim 3, wherein the processor is configured to set an imaging preset of a plurality of imaging presets based on user input for a scan session.

6. The wireless ultrasound probe of claim 5, wherein the processor is further configured to a set a rounds mode, wherein in the rounds mode a scan session is closed and a new scan session starts in response to a trigger event, wherein the closed scan session and the new scan session is associated with a patient.

7. The wireless ultrasound probe of claim 6, wherein the trigger event is based on user input.

8. The wireless ultrasound probe of claim 6, wherein the trigger event is in response to the processor receiving and processing an identification code associated with the patient.

9. The wireless ultrasound probe of claim 6, wherein the processor is further configured to:
determine presence of a previous scan session of the patient;
set at least one imaging preset of the previous scan session in response to starting a new scan session on the patient; and
present scan data associated with the previous scan session and the image data captured in the previous scan session.

10. An ultrasound imaging system comprising:
an image processing unit for processing a plurality of images; and
a plurality of wireless ultrasound probes configured to wirelessly communicate with the image processing unit, wherein a wireless ultrasound probe of the plurality of wireless ultrasound probes comprises:
an image capturing unit for capturing image data associated to an anatomy of a patient;
a processor configured to end a scan session once image data associated with an anatomy of a patient is captured and start a new scan session for capturing image data associated to an anatomy of another patient; and
a wireless communication unit configured to wirelessly communicate with an image processing unit, the wireless communication unit facilitate transfer of the image data to the image processing unit.

11. The ultrasound imaging system of claim 10, wherein the wireless communication unit is configured to communicate over one of a Wi-Fi network and a Bluetooth network.

12. The ultrasound imaging system of claim 10, wherein each wireless ultrasound probe of the plurality of wireless ultrasound probes communicate simultaneously with the image processing unit through a wireless channel.

13. The ultrasound imaging system of claim 12, wherein the scan session ends based on user input, the user input is received based on at least one of activation of a switch in the wireless ultrasound probe, a gesture and a motion gesture of the wireless ultrasound probe.

14. The ultrasound imaging system of claim 12, wherein the image data captured in the scan session is transferred to the image processing unit prior to starting the new scan session.

15. The ultrasound imaging system of claim 12, wherein the processor is configured to set an imaging preset of a plurality of imaging presets based on user input for a scan session.

16. The ultrasound imaging system of claim 12, wherein the processor is further configured to a set a rounds mode, wherein in the rounds mode a scan session is closed and a new scan session starts in response to a trigger event, wherein the closed scan session and the new scan session is associated with a patient.

17. The ultrasound imaging system of claim 16, wherein the trigger event is based on user input.

18. The ultrasound imaging system of claim 16, wherein the trigger event is in response to the processor receiving and processing an identification code associated with the patient.

19. The ultrasound imaging system of claim 16, wherein the processor is further configured to:
determine presence of a previous scan session of the patient;
set at least one imaging preset of the previous scan session in response to starting a new scan session on the patient; and
present scan data associated with the previous scan session and the image data captured in the previous scan session.
